(19) United States
(12) Patent Application Publication
(10) Pub. No.: US 2017/0349877 A1
(43) Pub. Date: Dec. 7, 2017

(54) CELL CULTURE SUBSTRATE FOR TRAIT INDUCTION OF NERVE CELL, METHOD OF CONTROLLING TRAIT OF NERVE CELL, METHOD OF EXTENDING NEURITE, METHOD OF CONTROLLING DOPAMINE SECRETION, AND METHOD OF CONTROLLING ACETYLCHOLINESTERASE ACTIVITY

(71) Applicant: Tokyo Ohka Kogyo Co., Ltd., Kanagawa (JP)

(72) Inventors: Emi MAENO, Kawasaki-shi (JP); Takahiro SENZAKI, Kawasaki-shi (JP); Atsushi MUROTA, Kawasaki-shi (JP); Yasuhiko TABATA, Kyoto (JP)

(73) Assignee: Tokyo Ohka Kogyo Co., Ltd., Kanagawa (JP)

(21) Appl. No.: 15/610,719

(22) Filed: Jun. 1, 2017

(30) Foreign Application Priority Data
Jun. 2, 2016 (JP) 2016-110925

(51) Int. Cl. C12N 5/0793; C12N 5/00

(52) U.S. Cl. CPC C12N 5/0619; C12N 5/0068; C12N 2535/10

(57) ABSTRACT

Provided is a cell culture substrate for trait induction of a nerve cell, which has a pattern of unevenness on a surface to which a cell adheres, the width of the unevenness being 50 nm or more and 1,000 nm or less.

### BACKGROUND OF THE INVENTION

### Field of the Invention

The present invention relates to a cell culture substrate for trait induction of a nerve cell, a method of controlling trait of a nerve cell, a method of extending a neurite, a method of controlling dopamine secretion, and a method of controlling acetylcholinesterase activity.

Priority is claimed on Japanese Patent Application No. 2016-110925, filed on Jun. 2, 2016, the content of which is incorporated herein by reference.

### Background Art

It is known that dopamine (DA) and acetylcholine (ACh) serving as neurotransmitters are in a relationship to antagonize each other, and when the amount of dopamine increases, the total acetylcholine decreases, and conversely when the amount of acetylcholine increases, the amount of dopamine decreases.

In Parkinson's disease (PD), it is considered that a DAergic neuron terminal projected from a substantia nigra compacta progressively decreases, so that ACh dominates relatively in a dorsal striatum (caudate nucleus or putamen), and a balance between DA and ACh is lost. A cause of the decrease in DAergic neuron terminal is not clear, and it is known that DA decreases by approximately 10% at every 10 years of age, and PD develops in old age. In PD patients, DA decreases and ACh increases, so that movement disorder appears.

On the other hand, it is clear that ACh decreases in Alzheimer's type dementia occupying 50% of dementia. ACh is known to act as a neurotransmitter for learning, memory, arousal, and sleep in the brain. In an early stage of Alzheimer's type dementia, short-term memory impairment is considered to be due to a decrease in ACh. In addition, it is common to increase acetylcholine with a therapeutic agent that inhibits the action of acetylcholinesterase (AChE) serving as an enzyme which degrades ACh.

In recent years, it has been expected that a neural stem cell or an embryonic stem cell (ES cell) is utilized to regenerate a nerve cell as a treatment for central nervous system diseases such as Parkinson's disease and Alzheimer's type dementia (for example, refer to Japanese Unexamined Patent Application, Publication No. 2004-357543).

### SUMMARY OF THE INVENTION

In a method described in Japanese Unexamined Patent Application, Publication No. 2004-357543, there is a problem that embryonic stem cells or the like are difficult to obtain (collect). In addition, even if the stem cells are directly transplanted to an affected area, the stem cells hardly differentiate into nerve cells and it is difficult to engraft. Even in a case where the stem cells are engrafted, most of these differentiate into glial cells, and differentiation cannot be controlled.

The present invention has been made in view of the above circumstances, and an object thereof is to provide a cell culture substrate that easily and efficiently induces a trait of a neural precursor cell in a certain direction.

The present inventors paid attention to the fact that trait induction of a nerve cell is due to the stimulation by nanoscale molecules on the cell surface, thereby completing the present invention.

That is, the present invention includes the following aspects.

According to a first aspect of the present invention, there is provided a cell culture substrate for trait induction of a nerve cell, which has a pattern of unevenness on a surface to which a cell adheres, the width of the unevenness being 50 nm or more and 1,000 nm or less.

According to a second aspect of the present invention, there is provided a method of controlling trait of a nerve cell, including culturing a neural precursor cell on the cell culture substrate for trait induction of a nerve cell.

According to a third aspect of the present invention, there is provided a method of extending a neurite, including culturing a neural precursor cell on the cell culture substrate for trait induction of a nerve cell.

According to a fourth aspect of the present invention, there is provided a method of controlling dopamine secretion, including culturing a neural precursor cell on the cell culture substrate for trait induction of a nerve cell.

According to a fifth aspect of the present invention, there is provided a method of controlling acetylcholinesterase activity, including culturing a neural precursor cell on the cell culture substrate for trait induction of a nerve cell.

According to the cell culture substrate for trait induction of a nerve cell of the present invention, the neural precursor cell can be easily and efficiently induced to a certain trait. In addition, neurite extension can be promoted and can be applied to regenerative treatment of nerves or treatment of central nervous system diseases such as Parkinson's disease and Alzheimer's type dementia. Furthermore, cells for drug discovery screening for the central nervous system diseases such as Parkinson's disease and Alzheimer's type dementia can be easily prepared.

### BRIEF DESCRIPTION OF THE DRAWINGS

FIG. 1A is an enlarged plan view and an enlarged front view of a cell culture substrate (pattern: line shape) for trait induction of a nerve cell according to an embodiment. FIG. 1B is an enlarged plan view and an enlarged front view of a cell culture substrate (pattern: dot shape) for trait induction of a nerve cell according to the embodiment.

FIGS. 2A and 2B are graphs illustrating measurement results of acetylcholinesterase (AChE) activity of PC12 cells in Example 1.

FIGS. 3A and 3B are graphs illustrating quantitative results of the amount of dopamine production of PC12 cells in Example 1.

### DETAILED DESCRIPTION OF THE INVENTION

Cell Culture Substrate for Trait Induction of Nerve Cell

In an embodiment, the present invention provides a cell culture substrate for trait induction of a nerve cell, which has a pattern of unevenness on a surface to which a cell adheres, the width of the unevenness being 50 nm or more and 1,000 nm or less.

According to the cell culture substrate for trait induction of a nerve cell of the present embodiment, a neural precursor cell can be easily and efficiently trait-induced to a certain direction. In addition, by using the cell culture substrate for trait induction of a nerve cell of the present embodiment, it is possible to induce differentiation from the neural precursor cell to the nerve cell, and further to control neurite extension, dopamine secretion or acetylcholinesterase activity to induce the trait in the differentiated nerve cell. In other words, the method of the present embodiment is a method of controlling the proliferation, differentiation and transformation of a nerve cell. Therefore, in the present specification, “trait induction” includes “induction of differentiation”.

Nerve Cell

In the present specification, “neural stem cell” refers to a stem cell that has multipotency capable of not only self-replicating ability but also differentiating into any of a neuron and a glial cell (for example, astrocyte, oligodendrocyte, or the like) serving as a main cell type constituting a central nervous system. Slightly differentiated precursor cell with self-replicating ability divides from the neural stem cell. The nerve cell is generated from the neural precursor cell, and the glial cell is generated from a glial precursor cell.

The cell culture substrate for trait induction of a nerve cell of the present embodiment can easily and efficiently induce differentiation of the neural precursor cell into the nerve cell (neuron) or differentiation maturation of the nerve cell.

In the present specification, “nerve cell (neuron)” includes the neural precursor cell.

The cell culture substrate for trait induction control of a nerve cell of the present embodiment is used, so that the nerve cell can be a differentiated and matured nerve cell. As a characteristic of the differentiated and matured nerve cell, the neurite extension is promoted, and the amount of dopamine secretion level or the amount of acetylcholinesterase activity is further increased.

Substrate

In the cell culture substrate for trait induction control of a nerve cell of the embodiment, a substrate to be used has a pattern of unevenness on the base plate. In addition, the substrate is not particularly limited as long as the substrate is not deformed when culturing cells or by pretreatment such as sterilization treatment. The whole of the substrate may be composed of the same material, and the substrate may be composed of a pattern of unevenness made of different materials, and a base plate for supporting the pattern of unevenness.

Examples of the form of the substrate include a multi-well plate, a petri dish, and the like, on which any number of wells are disposed. Examples of the number of wells include 6, 12, 24, 96, 384, 1,536, or the like per plate.

Base Plate

In a case where the substrate may be composed of the pattern of unevenness made of different materials, and the base plate for supporting the pattern of unevenness, the material of the base plate is not particularly limited as long as the base plate is used for cell culture applications. More specific examples of the material of the base plate include glass, polyethylene terephthalate, polycarbonate, cycloolefin polymer, polydimethylsiloxane, polystyrene, and the like. By using these materials, autofluorescent materials can be reduced, and the cultured cells can be observed with a fluorescence microscope.

Uneven Pattern

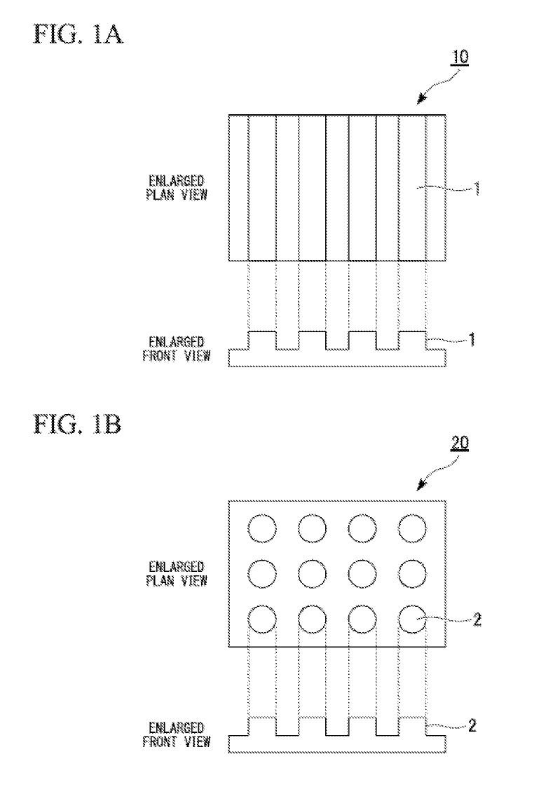

Examples of patterns of unevenness include a lattice shape, a radial shape, a polygon continuous shape on a flat surface (for example, a honeycomb structure or the like), a labyrinthine shape, a line shape, a dot shape, or the like. FIGS. 1A and 1B are an enlarged plan view and an enlarged front view of the cell culture substrate for trait induction control of a nerve cell according to the embodiment. FIG. 1A illustrates a case where the pattern of unevenness is a line shape, and FIG. 1B illustrates a case where the pattern of unevenness is a dot shape. In the enlarged front view of FIG. 1A, the shape of a projection portion is a rectangular parallelepiped line shape, but it is not limited thereto, and the shape of the projection portion may be a rectangular column shape (including a rectangular parallelepiped and a cube in a rectangular column), a truncated pyramidal shape, a semicircular column shape (including a semi-elliptical column in a semicircular column), a truncated conical shape (including an elliptical frustum and a biconical truncated cone in a truncated cone), or the like.

In addition, FIG. 1B illustrates a dot shape in which the transverse section of the projection portion is a circular shape and the vertical section of the projection portion is a rectangular shape (that is, the shape of the projection portion is circular column shape), but it is not limited thereto, and the transverse section or the vertical section of the projection portion may be a polygonal shape such as triangle and square, a circular shape (including a substantially circular shape, an elliptical shape, a substantially elliptical shape, a semicircular shape, and a fan shape in a circular shape), a trapezoidal shape, a wave shape, or the like. That is, examples of the shape of the projection portion include a rectangular column shape (including a rectangular parallelepiped and a cube in a rectangular column), a truncated pyramidal shape (including a truncated bipyramid in a truncated pyramid), a circular column shape (including an elliptic column, a semicircular column, a semi-elliptical column, and a sectoral column in a circular column), and a truncated conical shape (including an elliptical frustum and a biconical truncated cone in a truncated cone), or the like, but it is not limited thereto.

The width of the concave portion and the projection portion is preferably 50 nm or more and 1,000 nm or less, more preferably 75 nm or more and 1,000 nm or less, and further preferably 100 nm or more and 1,000 nm or less. When the width is within the above range, it is possible to stimulate the cell surface of the nerve cell and to easily and efficiently trait-induce the nerve cell to a certain direction. In a case where the shape of the projection portion is a circular column shape or a truncated conical shape, the width represents the diameter of the upper surface of the projection portion.

The distance to the surface of the projection portion of the uneven pattern is preferably 10 nm to 100 μm. In a case where the distance to the surface of the projection portion is within the above range, autofluorescence of the substrate is easily suppressed. Therefore, when the substrate having the distance within the above range is used, it is easy to observe the cultured cells by the fluorescence microscope.

Method of Forming Uneven Pattern

A method of forming the uneven pattern is not particularly limited. Examples of the method of forming the uneven pattern include a photolithography method in which a photosensitive composition layer formed on a surface of a substrate for supporting an uneven pattern is selectively exposed, and thereafter a portion corresponding to a concave portion is removed from the exposed photosensitive composition layer with a developing solution, an imprinting method of curing an imprint material after pressing a pressing mold having a pattern of unevenness on a layer of the imprint material formed on a base plate surface, a method in which a mask for covering a portion corresponding to a projection portion is provided on a base plate surface, and thereafter a concave portion is formed on the base plate surface by a chemical treatment such as etching, a method of grinding a base plate surface by sand blasting or various machine tools, a method of attaching a material constituting a projection portion of a pattern having a predetermined shape to a base plate surface, and the like. For the photolithography method and the imprinting method, a photosensitive resin composition used for various purposes in the related art and a photosensitive spin-on-glass (SOG) material can be used without particular limitation.

Photosensitive Resin Composition

Examples of the photosensitive resin composition used for forming the uneven pattern include a photosensitive resin composition containing a resin component, a cationic polymerization initiator, and a solvent, and the like. The photosensitive resin composition may be any of a positive type and a negative type.

The resin component is not particularly limited as long as the resin component can be used for cell culture, for example. Among these, a polymer of a compound having an ethylenic unsaturated bond is preferable. Examples of the polymerizable functional group contained in the compound having an ethylenic unsaturated bond include a (meth)acryloyl group, a vinyl group, an allyl group, and the like. As the compound having the ethylenic unsaturated bond, for example, a monofunctional, a difunctional, or a trifunctional or higher polyfunctional (meth)acrylate compound, a (meth)acrylamide compound, a vinyl compound, an allyl compound, or the like can be used. These compounds having the ethylenic unsaturated bond can be used alone or in a combination of two or more.

Examples of the polyfunctional compound having the ethylenic unsaturated bond include trifunctional or higher acrylates such as trimethylolpropane tri(meth)acrylate, pentaerythritol tetra(meth)acrylate, ethylene oxide modified pentaerythritol tetra(meth)acrylate, propylene oxide modified pentaerythritol tetra(meth)acrylate, dipentaerythritol penta(meth)acrylate, dipentaerythritol hexa(meth)acrylate, and the like; a polyfunctional urethane (meth)acrylate obtained by reacting a polyisocyanate compound and a hydroxy group-containing (meth)acrylate monomer; and a condensate of polyhydric alcohol and N-methylol(meth)acrylamide, and the like. These polyfunctional compounds can be used alone or in a combination of two or more.

Examples of the difunctional compound having the ethylenic unsaturated bond include polyethylene glycol di(meth)acrylate, polypropylene glycol di(meth)acrylate, polyethylene polypropylene glycol di(meth)acrylate, ethylene glycol di(meth)acrylate, propylene glycol di(meth)acrylate, polyethylene poly trimethylolpropane di(meth)acrylate, 2-(meth)acryloyloxy-2-hydroxypropyl phthalate, 2-(meth)acryloyloxyethyl-2-hydroxyethyl phthalate, a compound obtained by reacting a glycidyl group-containing compound with α,β-unsaturated carboxylic acid, urethane monomers, γ-chloro-β-hydroxypropyl-β′-(meth)acryloyloxyethyl-o-phthalate, β-hydroxyethyl-β′-(meth)acryloyloxyethyl-o-phthalate, β-hydroxypropyl-β′-(meth)acryloyloxyethyl-o-phthalate, and the like.

Examples of the compound obtained by reacting the glycidyl group-containing compound with α,β-unsaturated carboxylic acid include triglycerol di(meth)acrylate, and the like. Examples of the urethane monomer include addition reaction products of a (meth)acrylic monomer having a hydroxyl group at the β position with isophorone diisocyanate, 2,6-toluene diisocyanate, 2,4-toluene diisocyanate, 1,6-hexamethylene diisocyanate, or the like, EO modified urethane di(meth)acrylate, EO, PO modified urethane di(meth)acrylate, and the like.

Examples of monofunctional compounds having the ethylenic unsaturated bond include (meth)acrylic acid esters, (meth)acrylamides, allyl compounds, vinyl ethers, vinyl esters, styrenes, and the like. These compounds can be used alone or in a combination of two or more.

Examples of the (meth)acrylate esters include methyl (meth)acrylate, ethyl (meth)acrylate, propyl (meth)acrylate, amyl (meth)acrylate, t-octyl(meth)acrylate, chloroethyl (meth) acrylate, 2,2-dimethylhydroxypropyl (meth) acrylate, 2-hydroxyethyl (meth) acrylate, trimethylolpropane mono(meth)acrylate, benzyl (meth)acrylate, furfuryl (meth)acrylate, phenyl (meth)acrylate, (meth)acrylate of EO adduct of phenol, (meth)acrylate of PO adduct of phenol, (meth)acrylate of EO/PO co-adduct of phenol, ethylene glycol mono(meth)acrylate, diethylene glycol mono(meth)acrylate, triethylene glycol mono(meth)acrylate, polyethylene glycol mono(meth)acrylate, 2-methoxyethyl (meth) acrylate, diethylene glycol monomethyl ether mono(meth)acrylate, triethylene glycol monomethyl ether mono(meth)acrylate, polyethylene glycol monoethyl ether mono(meth)acrylate, propylene glycol mono(meth)acrylate, dipropylene glycol mono(meth)acrylate, tripropylene glycol mono(meth)acrylate, polypropylene glycol mono(meth)acrylate, propylene glycol monomethyl ether mono(meth)acrylate, dipropylene glycol monomethyl ether mono(meth)acrylate, tripropylene glycol monomethyl ether mono(meth)acrylate, polypropylene glycol monomethyl ether mono(meth)acrylate, mono(meth)acrylate of EO/PO copolymer, monomethyl ether mono(meth)acrylate of EO/PO copolymer, and the like.

Examples of the (meth)acrylamides include (meth) acrylamide, N-alkyl (meth) acrylamide, N-allyl (meth)acrylamide, N,N-dialkyl (meth)acrylamide, N,N-allyl (meth)acrylamide, N-methyl-N-phenyl (meth)acrylamide, N-hydroxyethyl-N-methyl (meth)acrylamide, and the like.

Examples of the vinyl ethers include alkyl vinyl ethers such as hexyl vinyl ether, octyl vinyl ether, decyl vinyl ether, ethylhexyl vinyl ether, methoxyethyl vinyl ether, ethoxyethyl vinyl ether, chloroethyl vinyl ether, 1-methyl-2,2-dimethylpropyl vinyl ether, 2-ethyl butyl vinyl ether, hydroxyethyl vinyl ether, diethylene glycol vinyl ether, dimethylamino ethyl vinyl ether, diethyl aminoethyl vinyl ether, butyl aminoethyl vinyl ether, benzyl vinyl ether, tetrahydrofurfuryl vinyl ether, and the like; vinyl allyl ethers such as vinyl phenyl ether, vinyl tolyl ether, vinyl chlorophenyl ether, vinyl-2,4-dichlorphenyl ether, vinyl naphthyl ether, vinyl anthranyl ether, and the like.

Examples of the vinyl esters include vinyl butyrate, vinyl isobutyrate, vinyl trimethyl acetate, vinyl diethyl acetate, vinyl valate, vinyl caproate, vinyl chloroacetate, vinyl dichloroacetate, vinyl methoxy acetate, vinyl butoxy acetate, vinyl phenylacetate, vinyl acetoacetate, vinyl lactate, vinyl-β-phenyl butyrate, vinyl benzoate, vinyl salicylate, vinyl chlorobenzoate, vinyl tetrachlorobenzoate, vinyl naphthoate, and the like.

Examples of the styrenes include styrene; alkyl styrene such as methyl styrene, dimethyl styrene, trimethylstyrene, ethylstyrene, diethylstyrene, isopropylstyrene, butylstyrene, hexylstyrene, cyclohexylstyrene, decylstyrene, benzylstyrene, chloromethylstyrene, trifluoromethylstyrene, ethoxymethylstyrene, acetoxymethylstyrene, and the like; alkoxystyrene such as methoxystyrene, 4-methoxy-3-methylstyrene, dimethoxystyrene, and the like; halostyrene such as chlorostyrene, dichlorostyrene, trichlorostyrene, tetrachlorostyrene, pentachlorostyrene, bromostyrene, dibromostyrene, iodostyrene, fluorostyrene, trifluorostyrene, 2-bromo-4-trifluoromethylstyrene, 4-fluoro-3-trifluoromethylstyrene, and the like.

The cationic polymerization initiator is one that generates a cation upon irradiation with radiation such as ultraviolet ray, far ultraviolet ray, an excimer laser such as KrF, ArF or the like, X-ray and electron beam, and the like, and the cation thereof is a compound that can be a polymerization initiator.

As the cationic polymerization initiator, for example, an onium salt type cationic polymerization initiator such as an iodonium salt or a sulfonium salt can be used. The anion to be a counter ion of the onium ion constituting the onium salt type cationic polymerization initiator is preferably a fluorinated alkyl fluorophosphate anion, a hexafluorophosphate anion, or a hexafluoroantimonate acid anion (SbF₆⁻).

The solvent contained in the photosensitive resin composition is not particularly limited as long as the solvent can prepare a uniform photosensitive resin composition and does not hinder the effect of exposure. The boiling point of the solvent is preferably 50° C. to 200° C.

Specific examples of the solvent include aliphatic hydrocarbons such as hexane, heptane, octane, decane, and cyclohexane; alcohols such as methanol, ethanol, 1-propanol, 2-propanol, and 1-butanol; acetone, methyl ethyl ketone, methyl isobutyl ketone, 2-heptanone, ethyl lactate, propylene glycol monomethyl ether acetate, propylene glycol monomethyl ether, ethyl acetate, butyl acetate, ethylene glycol dimethyl ether, propylene glycol dimethyl ether, methyl cellosolve, ethyl cellosolve, dibutyl ether, methyl-3-methoxypropionate, propylene glycol mono propyl ether, butyl cellosolve, diethylene glycol diethyl ether, hexylene glycol, cyclohexanone, propylene glycol monoethyl ether, ethyl pyruvate, ethyl cellosolve acetate, and the like. These may be used alone or in a combination of two or more.

In addition to the resin component, the cationic polymerization initiator, and the solvent, the photosensitive resin composition may contain various additives used in the photosensitive resin composition in the related art. Examples of such additives include additional resins, sensitizers, plasticizers, stabilizers, colorants, coupling agents, leveling agents, and the like.

The photosensitive resin composition can be prepared by mixing (dispersing and kneading) each of the above components with a stirrer such as a triple roll mill, a ball mill, a sand mill, or the like and filtering with a filter such as a 5 μm membrane filter if required.

Method of Preparing Substrate

The method of preparing the substrate is not particularly limited as long as it is a method capable of forming the substrate having a desired pattern by exposing and curing the above-described photosensitive resin composition. As a method of preparing a cell culture substrate, for example, a method including a coating process of coating a photosensitive resin composition onto a base plate to form a coating film and an exposure process of exposing the coating film on the base plate to cure the coating film can be mentioned. The method of preparing the substrate may include a detachment process of detaching the exposed coating film from the base plate after curing the coating film on the base plate by exposure if required.

In the coating process, the base plate on which the photosensitive resin composition is coated is not particularly limited as long as the base plate does not cause deformation or deterioration in the process of preparing the substrate. As the material of the base plate, the same base plate as the above “Base plate” can be mentioned.

As a method of forming a pattern of unevenness, the same method as the above “Method of Forming Uneven Pattern” can be mentioned.

The method of forming the coating film on the base plate is not particularly limited, and examples thereof include a method in which a predetermined amount of the photosensitive resin composition is dropped onto the base plate, a method of using a contact transfer type coating applicator such as a roll coater, a reverse coater, a bar coater, or the like, and a method of using a non-contact type coating applicator such as a spinner (rotary coating applicator), a curtain flow coater, or the like.

After the coating film is formed, the base plate provided with the coating film may be placed under a reduced pressure condition to degas the coating film.

In the exposure process, the method of exposing the coating film is not particularly limited as long as the coating film can be satisfactorily cured. For the exposure, for example, a light source emitting ultraviolet rays such as a high-pressure mercury vapor lamp, an ultrahigh pressure mercury vapor lamp, a xenon lamp, a carbon arc lamp or the like may be used. The exposure amount at the time of exposing the coating film is appropriately determined in consideration of the composition of the photosensitive resin composition, the film thickness of the coating film, and the like. Typically, the exposure amount when the coating film is exposed is preferably 10 to 100,000 mJ/cm², and more preferably 100 to 50,000 mJ/cm².

The method of exposing the coating film is not particularly limited, but the coating film may be first exposed to the atmosphere to partially cure the coating film. In this manner, in the exposure process, it is possible to prevent the photosensitive resin composition from protruding from the base plate, and thereafter to expose the coating film in water. If the coating film is exposed to water without exposure to the atmosphere, the coating film may dissolve in water in some cases. When the coating film is exposed to the atmosphere and thereafter the coating film is exposed to water, radical polymerization inhibition due to oxygen can be reduced and a good cured film can be obtained.

In addition, the exposure process may include exposure of the coating film in a vacuum. When the coating film is exposed to the vacuum, the coating film of the photosensitive resin composition can be cured in a state of being in close contact with the base plate, and a substrate having a desired pattern is easily formed. In addition, in a case where the coating film is exposed to the vacuum, exposure may be performed while applying pressure to the coating film from the upper surface of the base plate. In this case, the coating film of the photosensitive resin composition can be cured in a state of being in close contact with the base plate. When the exposure process includes exposure to the vacuum or exposure to the vacuum while applying pressure, specifically, in a case where a substrate is formed by using a mold corresponding to the pattern of unevenness provided in the substrate, it is possible to accurately transfer the uneven pattern of the mold to the substrate. By exposing the coating film under such conditions, shrinkage upon curing of the photosensitive resin composition is suppressed, so that the uneven pattern of the mold can be accurately transferred to the substrate.

As a method of exposing the coating film to the vacuum, for example, a method in which the surface of the coating film is coated with a film such as a PET film, and thereafter the coating film is exposed at least in a state where a space between the film and the coating film is vacuumed can be mentioned. In a case of exposing while applying pressure to the coating film, as a method of applying pressure to the coating film, for example, a method such as negative pressure exposure can be mentioned.

The coating film that is exposed and cured by the method as described above is used as the substrate after detaching from the mold if required.

In addition, the exposed and cured coating film may be subjected to a plasma treatment. By subjecting the cured coating film to the plasma treatment, it is possible to form the substrate to which the cell is likely to adhere. Plasma used for the plasma treatment is not particularly limited, but examples thereof include O₂ plasma, N₂ plasma, CF₄ plasma, and the like. The timing of the plasma treatment is not particularly limited, and the plasma treatment may be performed at any timing before or after detaching the cured coating film from the base plate.

Furthermore, the substrate detached from the mold may be rinsed with a rinsing liquid. When the substrate is rinsed with the rinsing liquid, a compound which can cause cytotoxicity such as an unreacted photopolymerizable monomer or photopolymerization initiator can be removed from the surface of the substrate. Examples of the rinsing liquid include organic solvents such as propylene glycol-1-methyl ether acetate (PGMEA), isopropyl alcohol (IPA), and acetone, water, and the like.

Application

In addition, a medical material with improved biocompatibility can be obtained by processing the cell culture substrate for trait induction control of a nerve cell of the embodiment on the surface of the medical material. Furthermore, by using the medical material, differentiation of the neural precursor cell can be controlled and tissues can be effectively regenerated.

Examples of medical materials include a medical molded body for scaffold used for tissue regeneration or transplantation tissue formation such as nerve and brain, and the like; a medical molded body for implanting in a living body such as an artificial nerve, and the like; surgical suture, surgical prosthetic material, surgical reinforcing material, wound protecting material, bone fracture bonding material, catheter, syringe, material for extracorporeal circulation, and the like, but the medical material is not limited thereto.

Method of Controlling Trait of Nerve Cell

In one embodiment, the present invention provides a method of controlling trait of a nerve cell in which a neural precursor cell is cultured on the cell culture substrate for trait induction of a nerve cell as described above.

According to the method of controlling trait of a nerve cell of the embodiment, the neural precursor cell can be easily and efficiently trait-induced to a certain direction. In addition, neurite extension can be promoted and can be applied to research on neural network formation or treatment of central nervous system diseases such as Parkinson's disease and Alzheimer's type dementia. Furthermore, cells for drug discovery screening for the central nervous system diseases such as Alzheimer's type dementia can be easily prepared.

In addition, the nerve cell differentiated from the neural precursor cell using the method of controlling trait of a nerve cell of the embodiment illustrates the neurite extension. The neurite extension can be a differentiation indicator.

In addition, in a portion of the nerve cells differentiated from the neural precursor cell, dopamine secretion capacity or acetylcholinesterase activity is increased.

Therefore, as described later, the method of controlling trait of a nerve cell of the embodiment can be used for applications such as “the method of extending a neurite”, “the method of controlling dopamine secretion”, or “the method of controlling acetylcholinesterase activity”, a differentiated nerve cell, and further a nerve cell having useful functions can be obtained.

Culturing Process

In the method of controlling trait of a nerve cell of the embodiment, the neural precursor cell is cultured using the above-described the cell culture substrate for trait induction of a nerve cell.

The neural precursor cell to be used is not particularly limited and may be a cell differentiated from the above-described neural stem cell.

The culture medium to be used may be a basic culture medium containing components (inorganic salts, carbohydrates, hormones, essential amino acids, non-essential amino acids, vitamins) and the like required for the cell's viable growth. Examples of the culture medium include Dulbecco's Modified Eagle's Medium (DMEM), Minimum Essential Medium (MEM), Basal Medium Eagle (BME), Dulbecco's Modified Eagle's Medium: Nutrient Mixture F-12 (DMEM/F-12), Glasgow Minimum Essential Medium (Glasgow MEM), Gibco® RPMI 1640 culture medium (manufactured by Life Technologies), and the like. In the culturing process, the culture medium may be suitably replaced with a new one according to the growth rate of the cells.

In addition, a compound inducing the differentiation of the nerve cell may be added to the culture medium to be used. By adding the compound, the rate of differentiation can be further accelerated, and differentiation can be controlled in a certain direction. Examples of compounds that induce differentiation of the nerve cell include nerve growth factor (NGF), dBu-cAMP, staurosporine, and the like, and two or more of these compounds may be used in combination.

The concentration of the compounds that induce differentiation of the nerve cell is not particularly limited, and may be 1 nM or more and 1 μM or less, and may be 5 nM or more and 500 nM or less. Within the above range, it is possible to more efficiently induce to differentiate from the neural precursor cell into the nerve cell.

Culture conditions are not particularly limited as long as it is a method suitable for culturing the nerve cell, for example, the density of seeding the nerve cell in the culture medium is preferably 1×10⁰ to 1×10⁷ cells/mL, and more preferably 1×10² to 1×10⁶ cells/mL. The culture temperature is preferably 25° C. or more and 40° C. or less, more preferably 30° C. or more and 39° C. or less, and further preferably 35° C. or more and 39° C. or less. The culturing time can be appropriately set depending on the growth state of the nerve cell, and it is preferably 1 hour or more and 100 hours or less. By using the cell culture substrate for trait induction control of a nerve cell as described above, the neurite extension and trait induction are promoted, and the nerve cell can be differentiated or trait-changed in a shorter time than the method in the related art. The culture environment is preferably cultured under CO₂ conditions through approximately 5% carbon dioxide.

Application

Method of Extending Neurite

In one embodiment, the present invention provides a method of extending a neurite in which the neural precursor cell is cultured on the cell culture substrate for trait induction of a nerve cell as described above.

According to the method of extending a neurite of the embodiment, the neurite can be easily extended and in a short time, and can be applied to the regenerative treatment of nerves.

Method of Controlling Dopamine Secretion

In one embodiment, the present invention provides a method of controlling dopamine secretion in which the neural precursor cell is cultured on the cell culture substrate for trait induction of a nerve cell as described above.

According to the method of controlling dopamine secretion of the embodiment, the amount of dopamine secretion in the nerve cell can be controlled and can be applied to the treatment of central nervous system diseases such as Parkinson's disease which is related to the amount of dopamine secretion.

Method of Controlling Acetylcholinesterase Activity

In one embodiment, the present invention provides a method of controlling acetylcholinesterase activity in which the neural precursor cell is cultured on the cell culture substrate for trait induction of a nerve cell as described above.

According to the method of controlling acetylcholinesterase activity of the embodiment, the production amount of acetylcholine in the nerve cell can be controlled and can be applied to the treatment of central nervous system diseases such as Alzheimer's type dementia which is related to the production amount of acetylcholine.

### EXAMPLES

Hereinafter, the present invention will be described with reference to Examples, but the present invention is not limited to the following Examples.

### Preparation Example 1

### Preparation of Substrate by Directed Self-Assembly (DSA)

(1) 0.2 mL of a propylene glycol monomethyl ether acetate solution containing 2 wt % of a block copolymer (number average molecular weight 18,000-b-18,000) of polystyrene and polymethyl methacrylate was dropped on a smooth surface of one sheet of a 0.8 cm×0.8 cm glass base plate (manufactured by Hiraoka Specialty Glass Co., Ltd.) to form a coating film on the base plate. Subsequently, the glass base plate on which the coating film was formed was annealed at 240° C. for 60 seconds. Subsequently, the coated film was subjected to O₂ plasma treatment under conditions of a pressure of 40 Pa, a temperature of 40° C., an output of 50 W, a treatment time of 20 seconds, and an oxygen flow rate of 200 ml/min, using a plasma processing apparatus (TCA-3822, manufactured by Tokyo Oka Kogyo Co., Ltd.), and the polymethyl methacrylate portion was selectively dry-etched to obtain a substrate (LS1). In addition, except for using a block copolymer (number average molecular weight 49,000-b-21,000) of polystyrene and polymethyl methacrylate, the same treatment was performed on the smooth surface of one sheet of a 0.8 cm×0.8 cm glass base plate (manufactured by Hiraoka Specialty Glass Co., Ltd.) to obtain a substrate (P1).

(2) 0.2 mL of a 2 wt % propylene glycol monomethyl ether acetate solution of polystyrene (number average molecular weight 18,000) was dropped on a smooth surface of one sheet of a 0.8 cm×0.8 cm glass base plate (manufactured by Hiraoka Specialty Glass Co., Ltd.) and one sheet of a 0.8 cm×0.8 cm polyethylene terephthalate (PET) base plate (manufactured by Mitsubishi Chemical Corporation) to form a coating film on the respective base plates. Subsequently, the coated film was subjected to O₂ plasma treatment under conditions of a pressure of 40 Pa, a temperature of 40° C., an output of 50 W, a treatment time of 20 seconds, and an oxygen flow rate of 200 ml/min, using a plasma processing apparatus (TCA-3822, manufactured by Tokyo Oka Kogyo Co., Ltd.) to obtain substrates (Smooth 1 and Smooth 2).

TABLE 1

| | Smooth | LS | P | Smooth | Smooth | P | P | P | P | LS | LS | LS | LS | LS | LS | LS |
|---|---|---|---|---|---|---|---|---|---|---|---|---|---|---|---|---|
| | 1 | 1 | 1 | 2 | 3 | 2 | 3 | 4 | 5 | 2 | 3 | 4 | 5 | 6 | 7 | 8 |
| Type of | Glass | | | PET | | | | | | | | | | | | |
| base plate | | | | | | | | | | | | | | | | |
| Photosensitive | Polystyrene | | | | Acrylic resin | | | | | | | | | | | |
| resin composition | | | | | (Radical negative resist) | | | | | | | | | | | |
| Pattern | — | Line | | — | — | Pillar | | | | Line and Space | | | | | | |
| | | and | Pillar | | | | | | | | | | | | | |
| | | Space | | | | | | | | | | | | | | |
| Width (nm) | 0 | 14 | 20 | 0 | 0 | 100 | 200 | 300 | 500 | 75 | 150 | 200 | 250 | 300 | 500 | 1,000 |

In Table 1, “Smooth” represents a flat substrate used as a control, on which an uneven pattern is not formed. In addition, “Width” represents the width of the projection portion.

### Preparation Example 2

### Preparation of Substrate by Transfer from Argon Fluoride (ArF) Pattern

A radical polymerization negative resist containing a photosensitive resin composition containing an acrylic resin as a main component was used as a photoresist composition. 1 ml of the radical polymerization negative resist was dropped on a 0.8 cm×0.8 cm silicon wafer having a pattern of unevenness (Smooth 3, P2 to P5, and LS2 to LS8) illustrated in Table 1 formed using ArF exposure machine Nikon S308, the coating film was degassed by placing the coating film under a reduced pressure condition of 100 Pa for 30 minutes, and a radical polymerization negative resist was embedded in the uneven pattern. Subsequently, twelve silicon base plates each having the coating film were exposed to an atmosphere with an exposure amount of 999 J/m² using an ultraviolet irradiation device (HMW-532D, manufactured by ORC Co., Ltd). Subsequently, the film cured by exposure as described above was covered with a base plate prepared by coating a radical polymerization negative resist having a film thickness of 1 μm onto a PET base plate (manufactured by Mitsubishi Chemical Corporation) so that a radical polymerization negative resist having a film thickness of 1 μm was in contact with the film cured, and exposure with an exposure amount of 999 J/m² was repeated five times using an ultraviolet irradiation device (HMW-532D, manufactured by ORC Co., Ltd) in a vacuum, to cure the coating film and the radical polymerization negative resist having a film thickness of 1 μm. After detaching the mold from the cured coating film, the cured coating film was immersed in propylene glycol-1-methyl ether acetate (PGMEA) for 10 minutes and rinsed, and thereafter the nitrogen gas was blown onto the cured coating film to be dried. Subsequently, O₂ plasma treatment was performed on the dried cured coating film under conditions of a pressure of 40 Pa, a temperature of 40° C., an output of 50 W, a treatment time of 20 seconds, and an oxygen flow rate of 200 ml/min, using a plasma processing apparatus (TCA-3822, manufactured by Tokyo Ohka Kogyo Co., Ltd.) to obtain a substrate (Smooth 3, P2 to P5 and LS2 to LS8).

### EXAMPLE 1

### Trait Control of Nerve Cell

(1) Culture of the Neural Precursor Cell

A cell culture test was performed at a culture temperature of 37° C. and 5% CO₂ environment using the substrate obtained in Preparation Examples 1 and 2. PC12 cells derived from rat pheochromocytoma (using RIKEN Bank cells, RIKEN Bank RCB 009) were used as the cell to be cultured. As a culture medium, DMEM high glucose medium containing 10% FBS was used. The substrate obtained in Preparation Examples 1 and 2 was placed in a well of a dish with a well, and thereafter 1×10⁴ cells per one substrate were seeded on the surface of the substrate. Thereafter, the culture medium was injected into the well with a disposable pipette and the culture for 1 day was cultured.

As a result of observing a form of the cells after the culture for 1 day, it was confirmed that the neurites appeared from PC12 cells in a case where substrates of LS3 to LS8 (line and space pattern, width of 150 nm to 1000 nm) and P2 to P5 (pillar pattern, width of 100 nm to 500 nm) were use.

(2) Differentiation of the Neural Precursor Cell

Subsequently, differentiation inducing compounds were added to a portion of cultured PC12 cells. Nerve growth factor (NGF) was used as a differentiation inducer.

As a culture medium, DMEM high glucose medium containing 10% FBS was used. The compounds were added so that the concentration of nerve growth factor (NGF) in the culture medium was 50 ng/mL and cultured for 2 days.

(3) Evaluation of Acetylcholinesterase (AChE) Activity

(3-1) Preparation of Cell-disrupted Liquid

A portion of the PC12 cells cultured in (2) was centrifuged at 200×g for 10 minutes at 4° C., and the supernatant was discarded. Subsequently, the cells were cleaned with 0.5 mL of phosphate buffered saline (PBS) and centrifuged again at 200×g for 10 minutes at 4° C. Subsequently, the cells were homogenized in 0.5 mL of assay buffer. Subsequently, the cells were centrifuged at 8,000×g for 10 minutes at 4° C. The obtained supernatant was used as a measurement sample of acetylcholinesterase activity.

(3-2) Measurement of Acetylcholinesterase (AChE) Activity

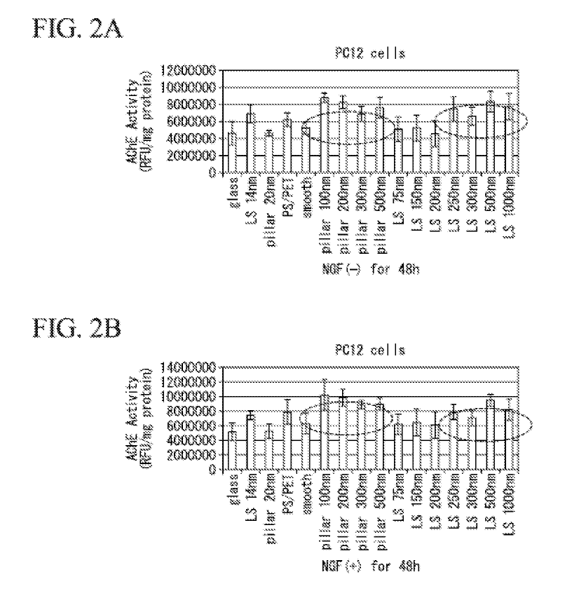

100 μL each of the measurement sample obtained in (3-1) and acetylcholinesterase (AChE) standard solution (0.025, 0.0125, and 0 units/mL) was added to the microplate. Subsequently, 50 μL of 2 mmol/L DTNB (5,5′-Dithiobis (2-nitrobenzoic acid)) solution and 50 μL of 2 mmol/L MATP+(1,1-Dimethyl-4-acetylthiomethylpiperidinium iodide) solution were added to each well and pipetted. As a control, wells in which 50 μL of a 2 mmol/L DTNB solution and 50 μL of an assay buffer were developed per 100 μL of the measurement sample were prepared. Subsequently, the wells were incubated at 37° C. for 30 minutes. Subsequently, the absorbance at 415 nm was measured using a microplate reader. Subsequently, the AChE activity in the measurement sample was determined from the calibration curve of the AChE standard solution. The results are illustrated in FIGS. 2A and 2B.

From FIGS. 2A and 2B, it was confirmed that AChE activity increased in PC12 cells cultured using P2 to P5 and LS5 to LS8 even in a case where NGF was not added.

(4) Evaluation of Amount of Dopamine

(4-1) Preparation of Cell Culture Supernatant

In (2), culture supernatants of cells cultured using each of the substrates were collected.

(4-2) Measurement of Amount of Dopamine

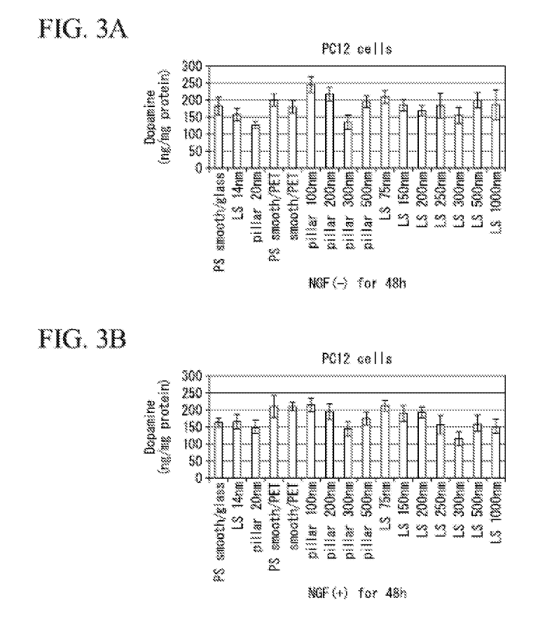

50 μL of each cell culture supernatant obtained in (4-1) was dispensed into wells of a microtiter plate on which mouse anti-DA antibody of Rat Dopamine (DA) ELISA Kit (manufactured by MyBioSource, Inc, Cat. No.: MBS 026032) was immobilized. In addition, 50 μL of each standard solution or a diluted solution of the cell culture supernatant was dispensed into each well. Subsequently, 100 μL of HRP conjugate reagent was dispensed into each well, the wells were covered, and incubated at 37° C. for 60 minutes. Subsequently, the microtiter plate was cleaned four times with 1×wash buffer (350 μL/well/single cleaning). Subsequently, the cleaning solution was removed, and 50 μL of the chromogen solution A and 50 μL of the chromogen solution B were dispensed into wells, respectively. Subsequently, the wells were gently mixed, incubated at 37° C. for 15 minutes with light shielding. Subsequently, 50 μL of the reaction termination solution was dispensed into each well, and the absorbance at 450 nm was measured using a microplate reader (Spectra Max i3 manufactured by Molecular Devices, LLC). The results are illustrated in FIGS. 3A and 3B.

From FIGS. 3A and 3B, it was confirmed that the amount of dopamine increased in PC12 cells cultured using P2, P3, P5 and LS 2, LS7 even in a case where NGF was not added. In addition, it was confirmed that the amount of dopamine decreased in PC12 cells cultured using P1, P4 and LS1, LS6 in the case where NGF was not added.

According to the cell culture substrate for trait induction of a nerve cell of the present invention, the neural precursor cell can be easily and efficiently trait-induced to a certain direction. In addition, neurite extension can be promoted and can be applied to the regenerative treatment of a nerve or treatment of central nervous system diseases such as Parkinson's disease and Alzheimer's type dementia. Furthermore, cells for drug discovery screening for the central nervous system diseases such as Parkinson's disease and Alzheimer's type dementia can be easily prepared.

What is claimed is:

1. A cell culture substrate for trait induction of a nerve cell, which has a pattern of unevenness on a surface to which a cell adheres, the width of the unevenness being 50 nm or more and 1,000 nm or less.

2. The cell culture substrate for trait induction of a nerve cell according to claim 1,
wherein the pattern of unevenness is a lattice shape, a radial shape, a polygon continuous shape on a flat surface, a labyrinthine shape, a line shape, or a dot shape.

3. A method of controlling trait of a nerve cell, comprising:
culturing a neural precursor cell on the cell culture substrate for trait induction of a nerve cell according to claim 1.

4. The method of controlling trait of a nerve cell according to claim 3,
wherein a culturing time of the nerve cell is 1 hour or more and 100 hours or less.

5. A method of extending a neurite, comprising:
culturing a neural precursor cell on the cell culture substrate for trait induction of a nerve cell according to claim 1.

6. A method of controlling dopamine secretion, comprising:
culturing a neural precursor cell on the cell culture substrate for trait induction of a nerve cell according to claim 1.

7. A method of controlling acetylcholinesterase, activity comprising:
culturing a neural precursor cell on the cell culture substrate for trait induction of a nerve cell according to claim 1.
